(19) United States
(12) Patent Application Publication
(10) Pub. No.: US 2023/0000261 A1
(43) Pub. Date: Jan. 5, 2023

(54) Pressurized Vertical Cylinder Air Chamber Mattress

(71) Applicant: Karen D. Webster, Memphis, TN (US)

(72) Inventor: Karen D. Webster, Memphis, TN (US)

(21) Appl. No.: 17/364,417

(22) Filed: Jun. 30, 2021

(51) Int. Cl. A47C 27/08; A47C 27/10

(52) U.S. Cl. CPC A47C 27/083; A47C 27/10; A61G 7/001

(57) ABSTRACT

An inflatable mattress that has multiple chambers is disclosed that includes a first section with a first pressure valve, and a second section that has a plurality of collapsible airtight vertical cylindrical chambers displaced across the first section and which defines a top surface and each vertical chamber includes valves, controlled by a central controller that is programmable and controls the flow of air in and out of each chamber, thereby providing a method to alter the vertical dimension of each chamber and affect movement to a person on the top surface mattress.

### INTRODUCTION

The present invention relates to an inflatable mattress system that can be automatically programmed with sequenced support positions or manually operated to adjust the support forces. By adjusting the support system, mattress occupants can be turned, and the mattress system provides other benefits such as massage effects.

### THE FIELD OF THE INVENTION

The present invention relates to the field of mattresses and mattress assemblies, and more specifically to air-filled mattresses having a plurality of cells. The degree and extent of fill of each cell, or groups of cells, in the mattress can be controlled either manually or may be programmed to provide predetermined inflation sequences. In embodiments, the particular cell and degree of inflation is designed to react to signals from biosensors integrated into the device. In embodiments the cells comprise vertically oriented airtight cylindrical chambers comprising valves to allow for air ingress and egress. The device also provides for the automatic adjustment of areas of support forces to individuals who are unable or only have a limited capacity to physically adjust themselves in bed.

### BACKGROUND OF THE INVENTION

In today's society, particularly with the advancement of medicine and engineering, with state-of-the-art computers, with discoveries in plastics and other products, and with advancement in electronics and other technology; there should also be an advancement in mattress structures to adequately care for the needs of world's growing population of seniors, as well as those with certain conditions or injuries, who need to be turned in bed.

The need for improved mattresses is particularly acute for elderly, the infirm and those in nursing home facilities, and those receiving home health care. Nearly 5 million people received care from a home health agency in 2013. People ages 75-84 (about 1.5 million) made up the largest share by age group of people receiving care from a home health agency. Nearly equal numbers (about 1.3 million) of people ages 65-74 and age 85 and over received home health care. See Older Americans 2016: Key Indicators of Well-Being. Federal Interagency Forum on Aging-Related Statistics Federal Interagency Forum on Aging-Related Statistics, Washington, D.C., U.S. Government Printing Office, August 2016.

In 2013, 1.3 million people received hospice care and nearly 50 percent (630,000) of the hospice patients were age 85 and over. See Older Americans 2016: Key Indicators of Well-Being. Federal Interagency Forum on Aging-Related Statistics, Washington, D.C., U.S. Government Printing Office. August 2016.

In 2014 it was reported that 29 percent of residents of residential care communities needed assistance transferring in or out of beds or chairs. See Older Americans 2016: Key Indicators of Well-Being, Federal Interagency Forum on Aging-Related Statistics, Washington, D.C., U.S. Government Printing Office, August 2016.

Persons confined to a bed not only include seniors, but also includes persons who have temporary medical conditions or long-term injuries that require bed confinement. Medical conditions that may require bed confinement can include spinal injuries, traumatic brain injuries, stroke victims, comatose patients, and patients with severe arthritis.

Regardless of the person, the ailment, or the cause for bed confinement; bed confinement can result in additional health-related issues for patients including both mental and physical conditions. Primary problems that result from being confined to a bed are skin shearing and pressure sores. With seniors in particular, issues from having pressure sores can be minor in type, e.g., a local infection, to major in type, e.g., an infection affecting major organs or requiring amputation. In some cases, problems from pressure sores can ultimately contribute to the senior's death. The Effects of Prolonged Bed Rest, Health 24, October 2013, https://www.health24.com/medical/sleep/treating-sleep-problems/effects-of-prolonged-bed-rest-20131025.

Bed sores can cause sepsis, a potentially fatal condition that occurs when bacteria from an open wound enters the bloodstream. Bed sores can also lead to bone and joint infections, causing septic arthritis and irreparable soft tissue damage. Bed sores can also cause the formation of cellulitis in the skin surrounding the sore, a condition that may eventually lead to the develop of life-threatening medical complications. See https://www.nursinghomelawcenter.org/what-is-turning-and-why-is-it-important-to-prevention-of-bed-sor.html.

Turning the patient is a crucial preventative treatment technique to avoid bed sores. The benefits of turning include the prevention of pressure ulcers, the improvement of oxygenation, and the decrease urinary stasis, all of which provide comfort to the patient. Frequent turning also provides improved regional ventilation in the lungs and allows alternating gravitational forces to drain mucus from sinus and lung cavities. Positional changes from turning tends to also decrease discomfort from being immobile, in particular back pain. Frequent turning is thought also to prevent the incidence of urinary tract infection. See Chris Winkelman, Ling-Chun Chiang, Manual Turns in Patients Receiving Mechanical Ventilation, Critical Care Nurse, 2010, August; 30(4):36-44. doi: 10.4037/ccn2010106. Further, frequently turning a patient also reduces susceptibility to other ailments, such as sleep apnea, which can then cause other serious conditions. See https://www.ncbi.nlm.nih.gov/pmc/articles/PMC3971026.

From 2002 to 2004 it was reported that death rates due to bedsores, also referred to as pressure ulcers, in the United States equal on average “34,319 per year, 2,859 per month, 659 per week, and 94 per day. This extrapolation calculation uses the deaths statistic: 34,320 deaths from decubitus ulcers were attributable to the patient safety incident in the US 2000-2002. See Patient Safety in American Hospitals, Health Grades 2004).” https://www.rightdiagnosis.com/b/bedsores/deaths.htm.

By 2012, the number of deaths caused from bedsores in the United States increased. “Researchers estimate that pressure sores are the proximate or originating cause of 60,000 deaths each year. The fatality rate for patients admitted to the hospital for treatment of pressure sores is around 13%. Recent studies have also demonstrated there is a causal link between bedsores and general patient mortality and prognosis. A 2012 study by researchers at UCLA Medical School found that seniors with pressure sores were statistically more likely to die after admission to a hospital (for any reason) than those without pressure sores. Elderly hospital patients that had pressure sores were also more likely to stay in the hospital longer and be back in the hospital sooner as compared to senior patients without pressure sores. The UCLA study was published in the Journal of the American Geriatrics Society.” See https://www.millerandzois.com/bed-sore-statistics.html.

Malpractice lawsuits due to improper wound care are not uncommon, thus it is every medical professional's responsibility to ensure that wounds are treated according to best practice. See: https://woundeducators.com/malpractice-wound-care/). The incidents of Medical Malpractice lawsuits due to improper wound care can be found at https://www.millerandzois.com/bed-sore-statistics.html. That site states that, “[P]ressure sores are a very unique type of medical injury in that they are almost always preventable. Even if a patient is completely immobile, pressure sores can be avoided with proper care and attention. This means that anytime a nursing home resident develops pressure sores, it automatically suggests that they did not receive the appropriate level of care. This is why pressure sores are frequently the basis for malpractice lawsuits against nursing homes and hospitals. Approximately 17,000 pressure sore lawsuits are filed in the US each year. This ranks pressure sores as one of the most frequently litigated injuries in the country. In contrast to other types of medical malpractice cases, data on pressure sore lawsuits suggests that plaintiffs prevail over 85% of the time. The national average for pressure sore lawsuit settlements is reported to be $250,000 (although this only accounts for publicly reported settlements).” https://www.millerandzois.com/bed-sore-statistics.html, referencing, Long-term Care Liability for Pressure Ulcers. See also https://www.ncbi.nlm.nih.gov/pubmed/16137292.

With respect to the overall cost of wound care, the cost as well as the incidents of wound care are increasing. “In the United States, chronic wounds affect 6.5 million patients. An estimated excess of US $25 billion is spent annually on treatment of chronic wounds and the burden is rapidly growing due to increasing health care costs, an aging population and a sharp rise in the incidence of diabetes and obesity worldwide. The annual wound care products market is projected to reach $15.3 billion by 2010. Chronic wounds are rarely seen in individuals who are otherwise healthy. In fact, chronic wound patients frequently suffer from “highly branded” diseases such as diabetes and obesity. This seems to have overshadowed the significance of wounds per se as a major health problem. For example, NIH's Research Portfolio Online Reporting Tool (RePORT; http://report.nih.gov/), directed at providing access to estimates of funding for various disease conditions does list several rare diseases but does not list wounds. Forty million inpatient surgical procedures were performed in the United States in 2000, followed closely by 31.5 million outpatient surgeries. The need for post-surgical wound care is sharply on the rise. Emergency wound care in an acute setting has major significance not only in a war setting but also in homeland preparedness against natural disasters as well as against terrorism attacks. An additional burden of wound healing is the problem of skin scarring, a $12 billion annual market. The immense economic and social impact of wounds in our society calls for allocation of a higher level of attention and resources to understand biological mechanisms underlying cutaneous wound complications.” See Human Skin Wounds: A Major and Snowballing Threat to Public Health and the Economy, https://www.ncbi.nlm.nih.gov/pubmed/19903300/.

Presently, the predominate method of turning a patient is manually. This process involves two abled assistants who stand on either side of the hospital bed (whether in a home, nursing facility, or hospital); and, if done properly with the use of a draw sheet, the assistants lift the patient slightly with the draw sheet, elevating the patient off the bed. Once in this position, one of the assistants pulls (the other keeps taut) the draw sheet, positioning the patient closer to the corresponding side of the bed. Then, the patient is rolled onto the patient's side toward the center of the bed. A wedge or pillows are used along the patient's back and hip to support the patient in that position. When it is time for another rotation, the action is repeated, but in reverse.

The problems that result from manually turning a patient include: (1) the attendants failing to timely turn the patient in order to prevent pressure sores; (2) the assistants are not strong enough to support the weight of the patient, causing the patient to be ‘dragged’ across the bed, the friction from which worsens the pressure sore; and (3) the problems associated with low pressure, i.e., skin breakdowns, are probable. As shown by the earlier statistics, these problems result in increased exposure for lawsuits at medical facilities, as well as increased costs for other public and private sectors in the United States.

Additionally, the mattress structures generally available to seniors or others who cannot turn themselves include:

Low or Alternating Pressure Mattress Structures. Low or alternating pressure structures redistribute points of contact between the occupant and the mattress by inflating and deflating sizable horizontal cells on the mattress that aid in blood flow to the occupant's skin. While these structures decrease skin and wound pressure problems, these products do not turn the occupant. The certified nursing assistants or personal medical assistants are required.

Turning Equipment. These structures assist with manual turning by allowing one attendant complete the turning action, e.g., U-Turner. While inexpensive, this type of equipment does not resolve the problems associated with manual turning, e.g., timeliness and friction. Additionally, the problem of high-pressure points on the occupant that come into contact with the bed is not resolved.

Horizontal Air Cell Mattress Structures. These structures provide for lateral rotation, however many do not have an ability to time the rotation, to set turn angles, or to make other adjustments. Some prior art solutions do not turn at all if head or foot of the bed is elevated. Some products offer preset rotation angles and cycles.

Egg-crate mattress toppers. Foam mattress toppers resemble an egg crate and contain craters that provide two alternating degrees of pressure to an occupant's body. The mattress toppers are used in conjunction with manual turning and consequently present the same issues associated therewith.

### OBJECTS OF THE INVENTION

In summary, the present invention is directed to providing an appropriate mattress for the aging, injured, and bedridden population that allows for easy turning of the mattress occupant. Accordingly, it is an object of the invention is to provide a mattress that assists and facilitates turning of persons who are bed ridden, but lack the ability, or have diminished ability to turn, adjust, or reposition themselves. It is a further object of the invention, to provide a mattress that can monitor vital statistics of an occupant including respiratory rate, temperature, pulse, and heart activity and automatically react to such signals by moving the occupant to mitigate potential adverse health consequences. It is a further object of the invention to provide a programmable control module that allows monitoring of the occupant and provides for both activation of air flow sequences at predetermined times and in response to sensors to cause to move an occupant of the mattress. It is further object of the invention to provide sensors on a mattress device to detect the location of the occupant on the mattress and provide an alarm in the event that occupant leaves the mattress. Other objects and features of the invention may be found in the detailed description of embodiments set forth herein.

### SUMMARY OF THE INVENTION

The mattress system of the invention is directed to a multi-layer programmable air mattress that includes at least one pressurized compartment that extends across the entire mattress structure. Embodiments include a lower support section, a middle section and an upper section. The upper section has a plurality of vertically oriented cylindrical chambers in which the pressure may be modulated in each chamber, or group of chambers. The mattress device of the present invention includes a turning feature, low pressure mode, head/foot raising feature, motion features including waves, vibrations, and massage. By controlling the air flow into the mattress chambers, the device can be programmed to perform varying degree of turns of an occupant over time, frequency of turns, and the timing of the turn cycle itself to be adjusted to the needs of the occupant. The invention also includes a network of microelectronic remote sensors that can detect the pressure, applied to the surface of the mattress, air pressure in the vertical air chambers and in the compartments of the mattress structure, as well as biometric readings including pulse and respiratory rate. As such the position of the occupant's body on the mattress structure as well as biometric data is detected. A control center receives and monitors signals from the remote sensors, operates the values and triggers the electric air compressor pump to make appropriate adjustments in the compartments of the mattress structure. In embodiments, bi-directional electronic valves affect the transfer of air to and from the vertical oriented cylindrical air chambers and a middle, pressurized compartment. The control center is remotely programmable with timed sequences. It can detect and manages the functions of the mattress structure and display numerical and graphic data regarding the mattress structure and the occupant. The controllers can also respond to the detection of data captured by the sensors.

### BRIEF DESCRIPTION OF THE DRAWINGS

The theory, substance, objective, and advantages of the specifications of the invention will be readily appreciated by the incorporated drawings and detailed descriptions provided below.

FIG. 1 is a perspective external view of the mattress structure.

FIG. 2 is a perspective view depicting the three internal sections of the mattress, referred to as the lower section, middle section and upper section.

FIG. 3 is a perspective view of a lower pressurized section.

FIG. 4 is a perspective view of the middle section showing bi-directional electronic air exchange valves and an external release valve.

FIG. 5 is a perspective view of the assembly that illustrates a number of vertically oriented cylinders displaced across a lower surface.

FIG. 6 is an exemplary display provided by a control center.

FIG. 7 is a schematic illustration depicting the elements of the improved mattress.

FIG. 8A is a view in elevation of the mattress depicting the vertically oriented cylinder air chambers in the upper pressurized compartment of the mattress structure in an exemplary stage.

FIG. 8B depicts a display element that illustrate the cell configuration of FIG. 8A.

FIG. 9 is a perspective view of an embodiment of a cell structure that characterized by a tubular sidewall in a first retracted or un-extended condition.

FIG. 10 is a perspective view of the cell structure of FIG. 9 in an extended condition.

FIG. 11 is a is a perspective view of a further embodiment of a cell structure that characterized by an accordion designed sidewall in a first retracted or un-extended condition.

FIG. 12 is a perspective view of the cell of FIG. 11 in an extended condition.

FIG. 13 is a side view in elevation of the mattress according to the invention.

FIG. 14 is a top view of a mattress wherein different zones are schematically represented.

FIG. 15 is a perspective view of an embodiment of the invention wherein a group of cylindrical chambers includes more than one inflow passage and a vent.

The foregoing drawings are provided for the purpose of pictorially describing the invention and are not intended to and should not limit the scope and applications of the invention.

### DETAILED DESCRIPTION OF THE EMBODIMENTS

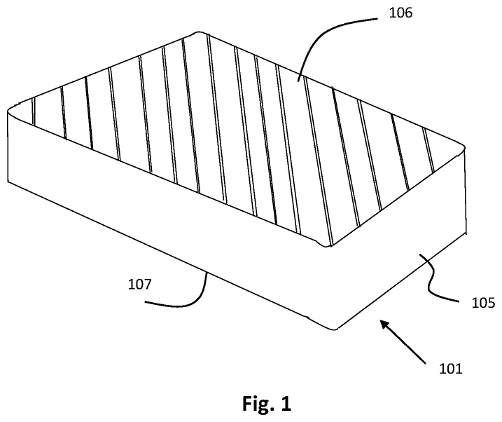

Now referring to FIG. 1, from the outside the smart mattress 101 according to the invention appears conventional and includes a sidewall 105, a top surface, 106 and bottom surface. While the dimensions can vary, in preferred embodiments the lower edge would contour over a standard hospital bed and have a dimension of approximately 36″ wide by 80″ long. The lower surface of the mattress has a plurality of flexible support elements integrated therein for firm support of the entire mattress structure. The mattress depicted in FIG. 1 has a cover that may include a zipper and is removable, lightly padded, and made of durable and washable bedding materials, which may provide cooling and additional comfort to the occupant. The external mattress cover houses and provides protection to the mattress structure.

The external mattress cover functions as a padding for the comfort of the occupant, as well as a protective sheath for the mattress structure, and may be constructed of a thin layer(s) of polyurethane foam, memory foam, latex (natural, synthetic, or a blend), polyester, feathers, wool, cotton, hempure, flax, or any combination thereof or of any other bedding material. The selected material should be sufficiently thick enough to be durable and to provide some comfort (pressure absorbency; softness; water-, heat-, and fire-resistance; non-toxicity; breathability) to the occupant, but thin enough for the microelectronic remote sensors at the top of the vertical air chambers in the upper pressurized compartment of the mattress structure to monitor the occupant's weight and position on mattress, as well as, the occupant's temperature and other biometric features that are be provided with the mattress according to the invention. The external mattress cover may be made to fit snuggly, encasing the mattress structure to allow the microelectronic remote sensors on the vertical pressurized chambers to function optimally. Also, the external mattress cover should have an open outlet to allow the electric compressor pump to be affixed to or inserted into the lower pressurized compartment, as well as an open outlet in the middle-pressurized compartment to allow the automatic release valve to function properly. The external mattress cover may also be removable by zipper or other type of closure for laundering (wiping) or for replacing for new occupants or for disposal when worn.

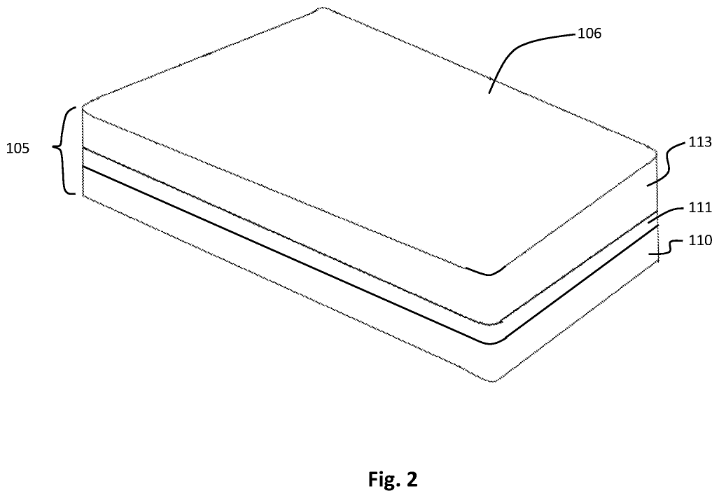

Referring now to FIG. 2 in an embodiment mattress 101 includes a lower section or compartment 110, a middle section or compartment 111 and an upper section or compartment 113. The upper compartment contains a plurality of separated chambers or cells (not shown). The mattress structure may be constructed of any plastic or rubber material or sheets (or combination of materials) that has been molded together (or any way constructed) that is strong enough to maintain its general shape under moderately pressures but is flexible enough to consistently and continuously longitudinally expanded at predictable amounts under controlled pressures. The upper section, including the vertical air chambers housed therein, are also strong but also are extremely flexible, thin, and collapsible. The selected material from which the chambers are made are nontoxic to humans and have characteristics suitable for use in hospitals and similar environments. Examples of such materials include, but are not limited to, PEEK, Ultem, Radel R, Polycarbonate, Polysulfone, Butyl Rubber, Hydrogenated Nitrile Rubber, Polypropylene, Ethylene Propylene Diene Monomer (EPDM), High-density Polyethylene (HDPE), and Graphene. Depending on the material, the entire mattress structure deflated may be collapsible to 5-7 inches in depth for better packaging and storage. Without pressurization, the upper pressurized compartment nestles in the middle-pressurized compartment, which nestles in the lower pressurized compartment. As an option, the entire assembly of the pressurized compartments may be bonded with a thin, sturdy, elastic material to maintain the proper contour, stability, and durability. Additionally, the bottom surface of the mattress structure and the side walls of the lower and middle pressurized compartments may be thicker or sturdier than that of the side walls of the upper pressurized compartment and the top surface of the mattress structure.

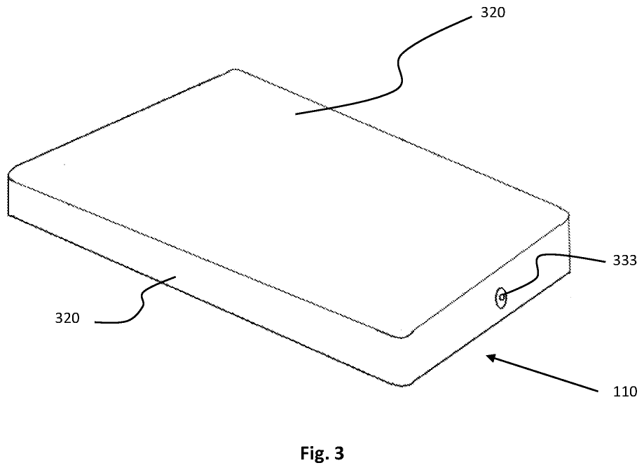

Referring now to FIG. 3, in this embodiment, a lower pressurized compartment 110 is provided that is primarily responsible for stabilizing and supporting the entire mattress structure, as well as the occupant while on the mattress structure. A bottom ridge of the lower pressurized compartment has a lip that snugly fits over the standard hospital bed frame (or box frame) to hold the mattress structure in place. Other embodiments can be constructed that have other dimensions and may be sized to fit on standard twin frames, queen frames, full frames and king frames. The lower pressurized compartment has an external duct 333, at which point an air pump hose attaches and transfers air from the air pressure pump to the lower pressurized compartment, and an electronic air transfer valve 320. Air transfer valve 320 allows air to be transferred from the lower pressurized compartment 110 into middle pressurized compartment 111. In embodiment the trander vale from compartment 110 to the middle section 111 is a one way valve. In embodiments, lower pressurized compartment 110 also has an internal electric remote sensor that measures and relays its pressure reading to the control center.

A constant pressure in the lower pressurized compartment 110 is maintained by the control center, which manages and maintains the optimal pressure depending on the weight of the occupant. Under normal operation, the air pressure in the lower pressurized compartment remains constant at all times during programming for any occupant. Air can be vented from mattress section 110 through valve 310 which in embodiments is pressure release value that opens after a predetermined pressure is exceeded or may be controlled by the central controller or be manually operated.

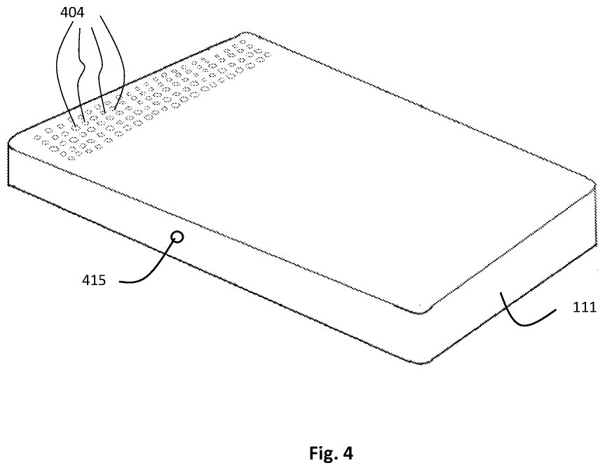

Now referring to FIG. 4, in the preferred embodiment, the middle-pressurized compartment's function is to act as an air-exchange unit, funneling air from the lower pressurized compartment to the vertical air chambers in the upper pressurized compartment. Another necessary function of the middle-pressurized compartment is to provide a flexible buffer zone in the mattress structure, enabling it not only to support the occupant, but also to allow all features of the mattress structure to function properly when the mattress structure is positioned at acute angles during scheduled programming. For example, when the head or foot of the bed is raised or when the occupant is positioned in a high angle turn, the buffer zone allows the mattress structure to keep the occupant balanced and allows the features of the mattress structure to operate. Thus, in embodiments, the relative depth or vertical dimension of the middle-pressurized compartment may depend on the features that are incorporated in the device.

The lower horizontal surface of the middle-pressurized compartment includes an air exchange valve (not shown) with the lower pressurized compartment 110 that allows air to enter the middle-pressurized compartment from the lower pressurized compartment. The middle-pressurized compartment also has sensors that measure internal pressure and transmits signals from the sensors to the control center. Along one of the side walls of the middle-pressurized compartment is an automatic release valve 415 that will discharge any surplus air accumulated during a previous stage in the programing or to collapse the mattress structure when the electronic control center is turned off. A transfer valve (one directional) is located in in the lower pressurized compartment to supply air to the middle-pressurized compartment.

Running along the top horizontal surface of the middle-pressurized compartment are the bi-directional air transfer valves 404 each of which connect with a vertical pressure cylinder chambers in the top section connect to the bottom of the vertical air chambers. These valves, which are displayed across the entire top surface of middle section 111, are similar to the standard air mattress, or any other bi-directional air transfer valve, except that the opening and the closing of these valves are controlled remotely and electronically by the control center. Thus, bi-directional air transfer valves will be opened and closed as needed to complete a particular position in the programmed sequences.

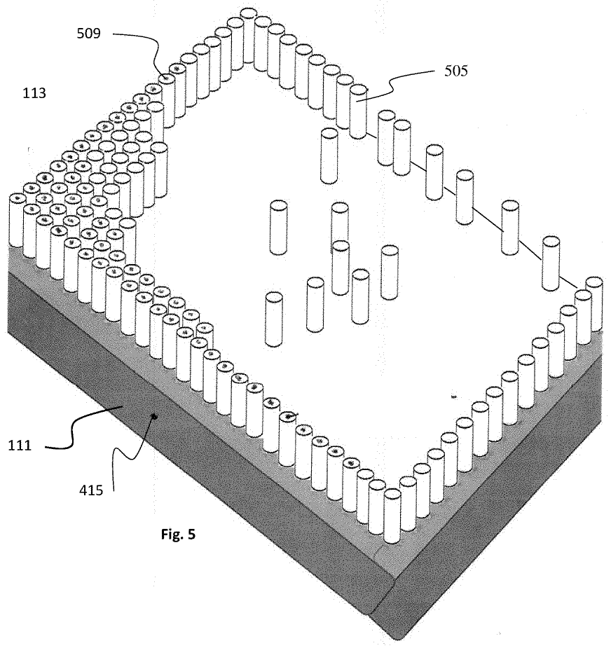

FIG. 5 illustrates fractional view an array of vertical chambers, such as chamber 505, that are provided across the top section 113 which is placed on middle section 111. While the illustration shows large areas without such chambers, in each of the embodiments the chambers are evenly distributed substantially continuously across the entire lower surface of the upper compartment. The vertical air chambers or cells provide the turning and low-pressure functions of the mattress structure and may also provide head and foot raising and lowering functions, as well other functions. The airflow provides the functionality of the vertical air chambers and is adjusted by the control center using the electronic air transfer valves connected to the upper pressurized compartment in conjunction with the surface pressure sensors such as sensor 509, provided in association with each vertical pressurized air chamber, or related group of chambers. The sensors continually measure the weight and position of the patent and transmit signals to the central controller.

The vertical air chambers are composed of a material that is extremely flexible and durable at high pressures.

The strength of the components of the vertical air chambers will allow the mattress structure to accommodate those of extreme heights and weights, given the appropriate size of the bed. Depending on the occupant, the vertical air chambers, particularly those running along the linear edges of the mattress structure, are designed to extend under pressure to a height of 9″-10″ or more in order to complete a high angle turn and will collapse to its original height of approximately less than one inch. The materials selected for the vertical air chambers allow them to expand vertically—at an angle of perpendicular to the bottom horizontal surface of the upper pressurized compartment—and restrict radial expansion. Thus, the vertical air chambers may be of multiple composites. In embodiment, the chambers or cells are comprised of air-tight polymer closed cells, such as latex, rubber, latex, polychloroprene, nylon, polyvinyl chloride, polyurethane or other urethane plastic and combinations of the same. In embodiments, the cells are contained by a lower membrane, sidewalls and an upper membrane. The number, shape, and size of the cylindrical air chambers or cells may be adjusted. In embodiments, the cells are contained in a pressured compartment. In embodiments some or all of the vertical air chambers have a sensor at the top of the chamber, and at the bottom of the chamber, and at least one electronically controlled valve to control airflow to and from the chamber. In some embodiments separate release valve is also provided.

Groups of vertical air chambers may be activated at the same time connected along a linear arrangement, may function independently, or be activated in other in preselected groups.

While the embodiments herein depict circular cylinders, the vertical air chambers may be formed in other shapes including cylinders having square top and bottom surfaces and flat sidewall, cylinders having polygon shaped top and bottom surfaces, cones, pyramids and frustums, some of which may provide more continuity or more dense arrangement of chambers in the mattress structure and may increase precision in turning functions.

The vertical air chambers may be any size or gradual sizes or any combination of shapes or sizes. Finally, the spacing between vertical air chambers may be constant or varied. Smaller and more compact the vertical air chambers will allow the mattress structure to provide a more fluid transition in the various motion functions provide by the cells. Also, certain shapes and sizes and their locations in the upper pressurized compartment will better benefit certain occupants, e.g., a design with smaller chambers on the outer lineal edges of the mattress structure will provide a quicker pressurization for the high angle turns.

As shown in FIG. 5, in an embodiment, the vertical air chambers are circular, spaced equal distances apart, work together jointly (connected along the linear surface(s) of the chambers), connected to the upper surface of the upper pressurized compartment), and are of a consistent circumference and length. The vertical air chambers all aligned at the lower surface of the upper pressurized chamber. Each vertical air chamber connects to a bi-directional electronic air exchange valve in the middle-pressurized compartment and allows air to be simultaneously forced back and forth between the middle-pressurized compartment and the vertical air chambers, as required in the sequences programmed in the electronic control center.

In embodiments, remote sensors are located at the top and bottom surface of each vertical air chamber. It is the function of the remote sensors at the bottom of the vertical air chambers near the electronic bi-directional air transfer valves to detect when the required air pressure is reached for that vertical air chamber. This information is relayed to the control center, at which time the corresponding electronic control center will close that bi-directional air transfer valve and will hold the pressure steady in that vertical air chamber until it is time to transition to the next sequence, as programmed in the electronic control center.

Running along the top surface of the upper mattress compartment over each of the vertical air chambers is a network of microelectronic that have several functions. The microelectronic remote sensors located at the top of each vertical air chamber communicate with the electric control center (1) to confirm the air pressure within that vertical air chamber; (2) to relay the horizontal position the occupant is laying on the mattress structure; (3) to measure the exact surface angle of that vertical air chamber, which operates to confirm the occupant's exact degree of rotation at that point on the mattress structure; (4) and to measure the temperature and other vital signs of the occupant. All information is transferred via the electrical network of microelectronic remote sensors to the electronic control center to determine the correct adjustment necessary to carry out the programmed schedule that has been set for the occupant.

Still referring to FIG. 5, when all of the vertical air chambers or cylinders are fully inflated to the same pressure, the cylinders have a consistent circumference and length, and are equally spaced across the middle compartment section.

As seen in FIG. 5, immediately above and affixed to the lower pressurized compartment is a middle, pressurized compartment, which also extends across the entire mattress structure. The upper top section of the mattress has a plurality of flexible members that comprises cylindrical air chambers which are in communication with the middle, pressurized compartment. The compartments are pressured with air using an electric air compressor. While flexible, the middle, pressurized compartment has a sturdy outer edge around the entire surface of the middle, pressurized compartment and is designed to maintains the minimal required pressure for supporting a person lying on the top layer. The middle, pressurized compartment also contains a remote sensor and a release valve to release pressure when the pressure in the middle section exceeds a predetermined level. It also includes a plurality of bi-directional electronic air transfer valves that are in communication with cell in the upper section. In alternative embodiments, the values are unidirectional, and a release value is provided on each cell that is controlled by the central controller that release pressure to the external environment. The release value may be designed to maintain a maximum pressure in the cell.

Immediately above, affixed to, and supported by the middle-pressurized compartment is the upper support section. Upper support section is comprised by an array of vertically oriented air chambers or cells, each chamber or cell comprising a circular cylinder, polygonal, or frustoconical shaped element that is designed to receive air and extend longitudinally.

In an embodiment, after activating the mattress, data relating an occupant's physical characteristics are entered into a central controller, including the occupant's weight and height. Optionally, positions that are required or recommend as part of treatment regimen for the occupant are also entered, such as elevation of the legs, and frequency of turning. Next, a fill sequence is initiated causing the activation of an air-pump which furnishes air to the lower pressurized compartment for the stability and support of the mattress structure. Once the desired pressure is reached, the lower pressurized compartment will maintain that constant air pressure at all times during programming for that occupant. After the preselected air pressure is established in the lower pressurized compartment, air is then directed to a middle-pressurized compartment and the section will inflate to the selected pressure designed to provide the support and stability of the occupant based on the height and weight of the occupant that has been entered into the control center. This middle chamber is also inflated at a threshold pressure so that it will provide the support needed the programmed positions and the proper functioning of the vertical air chambers in the upper pressurized compartment.

After both the lower and middle pressurized compartments are properly and adequately inflated, and before the upper pressurized compartment that contains the vertical air chambers begins its fill sequence, the mattress structure is in its “baseline rest” or stabilizing state. While in the baseline rest or a stabilizing state, the pressure in the middle and lower pressurized compartments also provide the necessary support for occupant during feeding, bathing, toileting, exiting the mattress and other daily activities. It is in this baseline state that a user may be placed or transferred on or off the mattress structure. For conventional programming, the air pressure at the baseline rest or stabilizing state will not change for that occupant. Regardless of the amount of air that is exchanged from the middle-pressurized compartment and forced into or released from the various vertical air chambers cells, the electrical remote sensor in the middle-pressurized compartment and air pump maintains the air pressure in the middle-pressurized compartment to remain constant for that occupant, stabilizing the mattress structure.

Once an occupant is on the mattress structure, the sequences programmed in the electronic control center can be started by pressing a ‘start program’ function. At the start of the initial sequence, the vertical air chambers in the upper pressurized compartment inflate from the air transferred by the bi-directional electronic transfer valves through the lower and middle pressurized compartments from the electronic air compressor pump. The elevation of or the pressure in the vertical air chambers increase gradually. The microelectronic remote sensors at both the top and bottom of the vertical air chambers, as well as the bi-directional electric air transfer valves connected to the bottom of the vertical air chambers work in tandem sending information to the electronic control center to properly manage the mattress structure. The elevation of or pressure in each individual vertical air chamber corresponds to the air pressure needed in that chamber to position the occupant while on the mattress structure, according to the positions in the sequences that was programmed into the electronic control center.

Once the vertical air chambers are properly inflated as programmed, all bi-directional electronic air transfer valves close, preventing air from being forced into its vertical air chamber and the electric compression pump automatically stops forcing air, allowing the middle and lower pressurized compartments to maintain the required constant pressure to maintain that position for that occupant. The mattress structure enters a rest stage. The duration of the rest stage may also be also programmed into the electronic control center.

While in the rest stage, the remote sensor at the top of each vertical air chamber (1) confirms the pressure contained in that vertical air chamber, (2) relays the pressure in each vertical air chamber to the control center, and (3) detects and transfers information about the occupant's vitals to the electronic control center. During the same time, the control center reads and continually monitors the sensors and all valves and manages the transfer of additional air into or out of the mattress structure through the electric compressor pump, if needed. The monitor on the electronic control center displays numerically and graphically information about the state of the system, including the upper surface configuration of the mattress structure.

After a rest stage has ended, the control center causes the mattress structure to begin the sequences for a next position. In embodiments, a bi-directional electronic air transfer valves will cause the cylindrical-shaped air chambers to begin to either inflate or deflate depending on the next position in the sequence that has been programmed in the control center. When the electronic control center detects the time for a position change, each bi-directional electric air transfer valve allows the airflow or exchange required at each interval in and out of the vertical air chambers and the middle-pressurized compartment. In this embodiment, the middle-pressurized compartment works as an air-exchange system, while maintaining its pressure relatively constant. In some embodiments, any additional air needed to reach the desired configuration is supplied from an air compressor pump through the lower pressurized compartment and excess air is discharged from a control release valve on the side of the middle-pressurized compartment to lower the pressure and thereby affect a new configuration. In yet alternative embodiments, the air is delivered to the upper compartment and its vertical air chambers from an air compressor using a separate manifest system which includes flexible hoses.

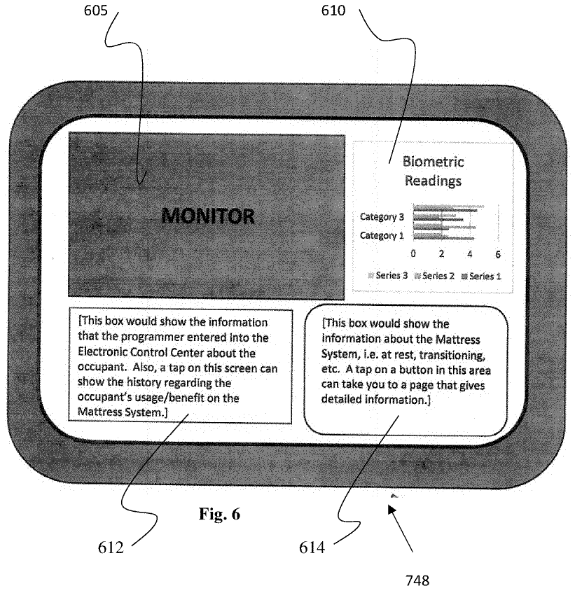

FIG. 6 depicts an exemplary display 748 that provides a plurality of field which include a visual representation of the mattress and occupant 605 (not shown), the data from biometric sensors 610, the occupants profile information including history 612, and information about the status of the mattress and fill sequence 614.

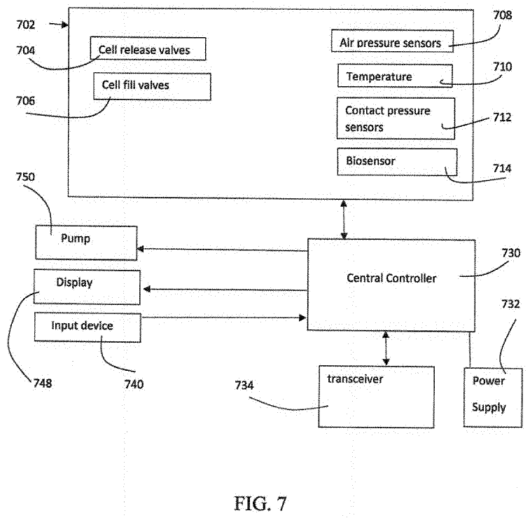

FIG. 7 is a schematic view of the controller and its communications with the smart mattress 702 including air pressure sensors, the vital sign sensors, the pump, the valves and the electronic display and the input device. The system includes a mattress structured 702 in which are provide a plurality or controllable pressure release values 704 for each cell or subset of cells, controllable cell inflow valves 706 to regulate the filling of cell or subsets of cells, air pressure sensors 708 to detect the pressure in cells, temperature sensors 710, contact pressure sensors 712, and biosensors 714. The cell fill valves, and cell release valves are controlled by a central controller 730. Central controller 730 takes input from input device 740 which, in embodiments, are a keyboard and mouse devices. In embodiments, input and output to the controller can also be received and transmitted through wireless transceiver 734 which allows for the both the monitoring and control of the mattress system from remote locations. While the schematic of FIG. 7 identifies both cell fill valves and cell pressure relief valves, in embodiments a bidirectional value can be used to perform both functions.

The control center controls the operation of the mattress structure and receives input from the sensors. The control center receives various categories of input from the programmer regarding the occupant and the desired functions that are to be provided. It further monitors the all of the components of the mattress structure and, in response to signals from the pressure sensors, maintains the designated pressure in the compartments of the mattress structure, reads signals from the sensors throughout the mattress structure, activates and shuts off the air compressor and controls the bi-directional electronic air-transfer valves to respond according to the programmed functions. The monitor on the control center provides numerical and graphic data regarding the programming, the operations of the mattress structure, and the occupant.

The control center controls the various functions of the mattress structure, including the operation of the electric air compressor pump. Using the control center, a user can program the turning sequences, the pressure of the mattress, and head/foot raising and lowering functions of the mattress structure; and the user may adjust the transition timing, air pressure, and degree of turn or elevation sequences for each function. In embodiments, predetermined default turning sequences are provided including transition times for varying weight, height, age, and other conditions of the occupant. These predetermined defaults settings may be manually adjusted by the user and any programming may be paused, stopped, stored, or overridden by subsequent programming. The programmed sequences in the control center can be either entirely user programmed settings or predetermined default settings. Either programmed setting or user defined settings may be manually overridden and later manually returned to the initial programmed sequences at any time.

In an embodiment, during operation, the control center (1) monitors and reads the microelectronic sensors provided on the upper horizontal surfaces of the vertical air chambers (2) controls and manages the functioning of microelectronic air transfer valves and other remote sensors; (3) controls and manages the air compressor pump, causing adjustments of air pressure increased or decreased (release) in the pressurized compartments, taking into consideration the weight of and the position of the occupant, as well as the position of the bed; (4) maintains optimal air pressure in the middle pressurized compartment to allow the free transfer air to the vertical air chambers; (5) controls the transfer of air (adjusting pressure) to and from each pressurized compartment; (6) and manages any other function of or feature provided with the mattress structure.

The control center uses the information collected from all remote sensors in the mattress structure to compare with the sequence programmed for the occupant to determine if adjustments are necessary. As adjustments are needed in any place within the mattress structure, the electronic control center signals the proper component to make the correction; e.g., if more air is needed in a particular vertical air chamber, the electronic control center signals the electric air compressor pump to furnish more air and the corresponding bi-directional air transfer valve is signaled to open; in order to properly carry out stabilizing, turning, or low pressure functions of the mattress structure as programmed. The electronic control center—always sensing the position of the occupant from the network of microelectronic remote sensors above the vertical air chambers—verifies the exact position (angle of turn) of the occupant and displays this information on its screen, both numerically and graphically.

In embodiment the control center has a computerized touch screen, which allows the user to program all the required functions for a given occupant. All information regarding the mattress structure is accessible and adjustable and may be displayed by the user while the mattress structure is turned on. The electric control center may be attached to the mattress structure, the electric air compressor pump or may be a remote center. In embodiments, the electric control center will not turn on the power for the electric air compressor pump, unless the electric air compressor pump is constructed as a permanent feature with the mattress structure.

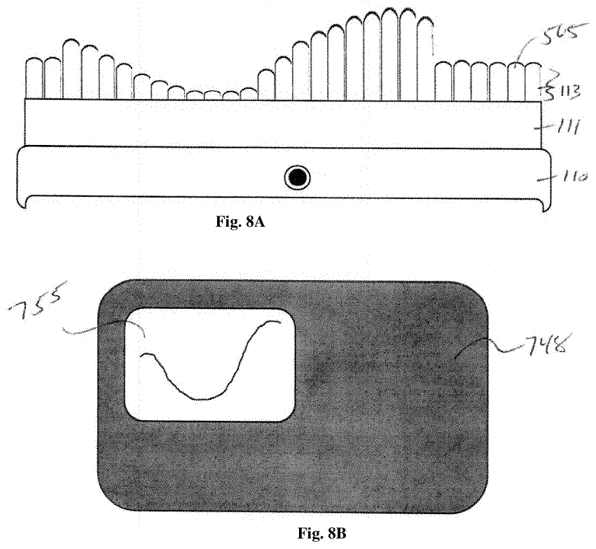

Referring now to FIG. 8A, the illustration in elevation depicts the initiation of a rotation from the standpoint of the foot of the mattress. FIG. 8B shows an exemplary display on the monitor of the electric control center corresponding to that position.

Once the process for the second position in the sequence is complete, the mattress structure will again be filled and return to the rest phase and equilibrium. This cycling continues over time until all the sequences in the programming have been completed. The control center manages all functions of the mattress structure, as programmed by the user.

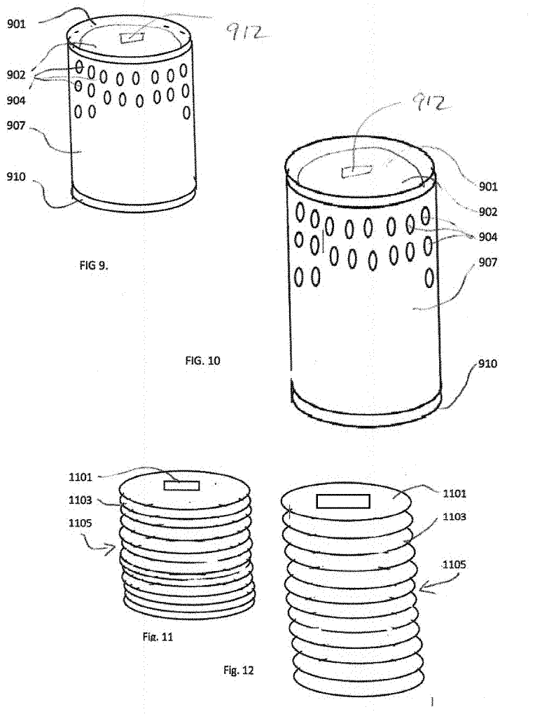

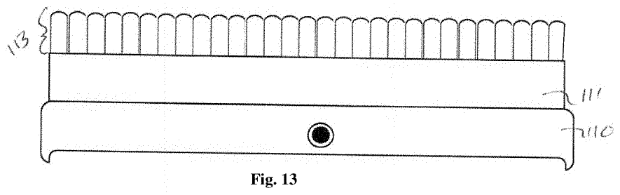

In a further contemplated embodiment as depicted in FIGS. 9 and 10, cylinders are provided in the form of cells that are comprised of flexible bladder 902 that are contained in a fabric cylindrical cage 907. Cages 907 allow for extension of the bladder in a vertical direction, transverse to a plane defined by the top of the mattress and sidewall of the cages 907 restrict radial expansion. As such when pressure is increased in a bladder, the bladder expands vertically from base 910 and the cell or chamber extends in a vertical direction, perpendicular to the planar surface on which they are arranged. The cages 907 have apertures, such as apertures 904 through sidewall 907 to facilitate the vertical expansion and retraction of sidewall 907. In other embodiments cages are comprise of a mesh fabric.

In a further contemplated embodiment as depicted in FIGS. 11 and 12, an elongate cylindrical-shaped chamber 105 is provide which comprise a structure with an accordion-shaped sidewall 1103 that enables the chamber to increase and decrease its respective vertical or longitudinal dimension in response to an increase in pressure, while restricting radial expansion. Sidewall 1103 is made from adjacent annular sidewall sections which create a mountain and valley arrangement. On the top surface of the chamber 1101 a sensor 1110 is provided

In yet a further embodiment the cells are comprised of bladders in the same shape of a tubular fabric sleeve that restricts lateral expansion but allows for expansion longitudinally transverse to a plane formed by the top of the mattress. For example, the relative sidewalls of the chamber may have varying thickness to provide bladder that extends longitudinally but to a lesser degree radially.

In yet a further alternate embodiment, the vertical air chambers have circular sections, work together jointly, connected at the top surface of the upper pressurized chamber, but of a gradual decreasing size from the center of the mattress structure. The vertical air chambers are at their longest circumference along the lengthwise middle of the mattress structure and decrease in circumference length gradually to the outer, side edges of the mattress structure, where they are at their shortest circumference. The vertical air chambers, all aligned at the bottom edge of the upper pressurized chamber, but only the vertical air chambers on one side may simultaneously receive air from the middle-pressurized chamber without the use of bi-directional electric air transfer valves.

During operation of the foregoing embodiment, air is supplied from the electric air compressor pump to and through the lower pressure compartment. As pressurized air is transferred from the middle-pressurized compartment and enters the right side, e.g., of the upper pressurized chamber of the mattress structure, the vertical air chambers having the smallest circumference will begin to elevate first, initiating the occupant's turn to the left. This process will continue until the occupant is completely turned to the left to the degree programmed in the electronic control center. The occupant will remain in this position for the time entered into the electronic control center. The outer edges of the left upper pressurized compartment will become a slightly heightened barrier to prevent the occupant from becoming lodged against the bed rails. All other features may be designed to function the same, except, some of the operation of the mattress structure in this embodiment is limited due to the inability to control the direction of the air at various junctions, e.g., the deflation of the vertical air chambers will depend on the passage of time before the next sequence function can begin.

In yet another embodiment the vertical cells are frustoconical in shape that facilitate expansion and collapse in a rectilinear direction.

In further embodiments, a program executed by the control center controls the inflation and deflation of cells follow predetermined sequences. In other embodiments the control of the inflation and deflation is based upon signals from the sensors integrated into the mattress which include pressure sensors and may include other sensor such as vital signs. For example, in an embodiment, if sensors detect a low pulse rate, the fill sequence may be altered to provoke a response by the mattress occupant try to increase the pulse. If the sensors detect a high local temperature, the inflation and deflation sequence may be altered to cause the user to move. In yet a further application, if the vital sensors detect a signal that reflects sleep apnea, the controller will initiate a sequence to wake the occupant and, inflate or delate cells in an attempt to open the airway. Data from the vital sensors can be displayed using visual and auditory signals including an audible alert to the occupant or those monitoring the occupant. In further embodiments, the data is transmitted to remote locations for remote monitoring, date storage or both.

In yet further embodiments, lowering and elevating the legs can be changed in response to blood pressure. Existing clinical studies showed that blood pressure is simultaneously controlled by the sympathetic nerve and parasympathetic nerve, and that heart rate is as an effective clinical metric to reflect the function of autonomic nerve system (the sympathetic nerve and parasympathetic nerve are collectively called the autonomic nerve system). Lutfi M. F., Sukkar M. Y. Effect of blood pressure on heart rate variability. Khartoum Med. J. 2011; 4:548-553. Hypertension, blood pressure, and heart rate variability: the Atherosclerosis Risk in Communities (ARIC) study. Schroeder E B, Liao D, Chambless L E, Prineas R J, Evans G W, Heiss G Hypertension. 2003 December; 42(6):1106-11. As such, it is possible to quantitatively investigate cardiac conditions by analyzing heart rate variability (HRV), including hypertension identification. See Ni H., Cho S., Mankoff J. Automated recognition of hypertension through overnight continuous HRV monitoring. Poddar M. G., Kumar V., Sharma Y. P., J. Ambient Intell. Hum. Comput. 2018; 9:2011-2023. doi: 10.1007/s12652-017-0471-y. Heart rate variability-based classification of normal and hypertension cases by linear-nonlinear method. Def. Sci. J. 2014; 64:542-548. doi: 10.14429/dsj.64.7867. For example, Poddar et al. disclosed a technique to identify normal and hypertensive cases using electrocardiogram (ECG) data and then characterized the fluctuation pattern of HRV. Others have extracted not only HRV-related features from photoplethysmography (PPG) data, but also many other features from galvanic skin response and skin temperature to model the hypertension patterns. See Detection of essential hypertension with physiological signals from wearable devices, Ghosh A, Tones J M, Danieli M, Riccardi G., Annu Int Conf IEEE Eng Med Biol Soc., 2015 August; 2015, pp 8095-8.

As described, the top section contains a plurality of elongate cylindrical-shaped airtight chambers of the mattress and this feature of the invention works in conjunction with a sensor array, electromechanical valves, and the processor. In some embodiments the cylindrical chambers are contained in a second pressurized top section. In embodiments, bi-directional valves having an elongate profile are provided to control airflow into and from the cylindrical chambers, wherein the valves extend into an adjacent lower chamber section. Embodiments of the invention have the ability to provide an automatic turning feature that can be programed to be implemented at predetermined speeds and frequency of cycle periods. In further embodiments, a program may be directed to a low pressure pre-set and programmable inflation patterns, head and foot raising and lowering features, each with variable ranges of elevation.

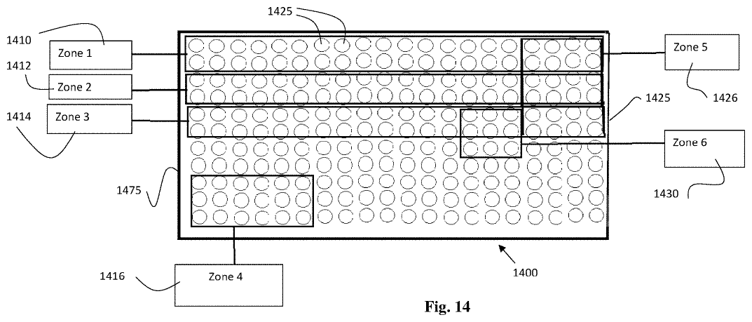

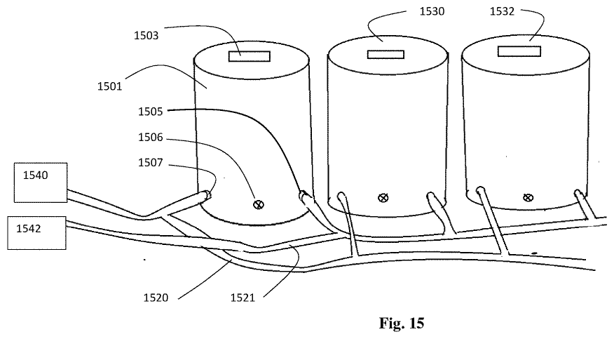

Now referring to FIG. 14, a top view of a mattress with the cylindrical chambers that illustrates the concept of providing different zones that having a plurality of chambers interconnected so that they may be simultaneously adjusted by the central controller. The mattress has a plurality of chambers, such as chambers 1425 displaced across the mattress. At a starting point, all of the chambers are adjusted at an equal pressure and the mattress has a uniform flat surface. Next the controller may case air pressure to be increased in zone 1, 1410 cause an occupant on the mattress to turn to the center. At the same ema time the controller may cause air to vent from zones 2 (Reference No. 1412) and Zone 3 (Reference No. 1413), causing an occupant of the mattress to turn toward the center longitudinal axis of the of the bed. In another sequence, Zone 2 and Zone 3 are reinflated. Also depicted is Zone 4 (Ref no. 1416) which may be inflated with other zones that border the edge 1475 to cause one end of the mattress elevate with respect to the center of the structure. Zone 6 (Ref. No. 1430) is adapted to be activated wherein all the cylindrical chambers are filled with air to increase their vertical dimensions and cause an occupant on the mattress to move and initiate a turn from the center of the mattress. Zone 5 may be included to cause the chamber therein to extend vertically and elevate that section either alone or in combination with other zones.

FIG. 15 is a perspective view of an embodiment where chambers 1501, 1530 and 1532 are attached to separate air fill systems 1540 and 1542. Chamber 1501 includes pressure sensor 1503. Fill system 1540 is attached to chamber 1501 at inlet passage 1507 and the system includes tubular section 1520 that also supplies chamber 1530 and 1532. Second system 1542 is connected to inlet passage 1505 of chamber 1501. It also supplies chambers 1530 and 1532. Each of the chambers has an exit valve such as exit valve 1506. In embodiments, a single valve, controlled by a central controller, is located at a stem location 1540 and controls the air fill in each of the chambers in a particular zone. In embodiments, air hoses 1520 and 1521 are pressure hoses

The present invention is accordingly directed to an improved mattress assembly that may be programmed to automatically adjust elastic support forces using an air compressor pump and a plurality of chambers each with its air pressure regulated by bi-directional electronic controlled air-transfer valves. The assembly includes sensors to detect the air pressure in each cylindrical chamber and surface pressure applied on the top surface. This network of sensors is designed to measure the occupant's weight distribution, temperature, and other metrics such as pulse, and respiratory rates.

The cylinders provide adjustable support using air that flows in and out of the cylinders through control valves. The air pressure is maintained at a constant level in a lower section by an electric air compressor pump. The electric air compressor pump may be constructed as a permanently connected component of the mattress structure, unattachable but provided with the mattress structure, or may be a commercially available pump that that can be e integrated into a computer-controlled system.

By detecting the location of the mattress occupant and then lowering the pressure in selected cylinders at locations adjacent to the occupant, the mattress can cause the occupant to turn. In addition, the system allows for the adjustment of air pressure and related cylinder height at zones in the mattress to elevate or lower an occupant's head and feet.

In an embodiment, in operation, the electronic control center is activated and next the weight, age, and height of the occupant is entered into electronic control center. At this point, the user may also program the turn and other motion sequences needed or desired to benefit the occupant into the electronic control center; or alternatively, the sequence programming may be entered once the occupant is on the mattress structure. In yet other alternative operations, the user can select pre-programed motion sequences that are appropriate for the mattress occupant. In yet further embodiments, the pressure in the cylindrical chamber is adjusted by the central controller in response to signals from pressure sensors on said mattress surface, signals from biometric sensors on said mattress surface or both.

In custom programming sequences, the user programs the specifics of the turns and other motion sequences (degree or angle, duration, number, duration of transition periods, duration of rest periods, etc.) that are needed for the occupant as ordered or directed by the physician or as desired.

Once the programming is complete, the user activates the ‘inflate’ switch. The control center then causes the air compressor pump to force sufficient air into the lower pressurized compartment 110 to properly support all functions of the mattress structure, given the weight, age, and height of the occupant. After the lower pressurized compartment is adequately inflated, the middle-pressurized compartment 111 inflates and maintains the required pressure for the mattress structure to enter a ‘baseline’ rest position, given the occupant's weight, age, and height.

At this point, the mattress structure will be in the ‘baseline’ resting position. From an external view, the mattress structure looks like any other mattress, at this point in the sequence and an occupant can be placed on or removed from the mattress structure or allowed to access or get off the mattress structure. Once the occupant is positioned in the center of the mattress structure, the programmed sequences may be started by activation of the program.

Whether or not the ‘start program’ command has activated, the occupant's vital measurements are taken when the occupant is in the center of the mattress structure and the electronic control center detects that no additional adjustments are being made through the electric remote sensors. Additionally, in embodiments, each sensor at the top of the vertical air chamber reads the occupant's proportional weight distributed across that vertical air chambers. The electronic control center will use this weight distribution information in calculating the pressure needed (or the height required) in the vertical air chambers to manage and complete each interval of the sequence programming entered by the user. If the program has started, the programmed sequence will initiate once the vital and distribution information has been received. Otherwise, the initiation of the air fill may be commenced at this point by activating an air fill sequence command.

The operation of the mattress structure in a right turn, e.g., might be as follows: (1) Once the program is started, additional air is forced into the mattress structure from the air compressor pump to the lower pressure compartment 110 and then to the middle-pressurized compartment 111. Using the additional air, as the pressure in both the lower and middle pressurized compartments will remain constant, the required bi-directional electronic air transfer valves at the upper horizontal surface of the middle pressurized compartment (connected to the lower horizontal surface of the vertical air chambers) are next opened and allow for the transfer air into designated vertical air chambers, causing them to become pressurized and to elevate, initiating a first position in the sequence programming entered for the occupant. As the turn starts (turning the body to the right), the vertical air chambers on the immediate right side of the occupant, as detected by the sensors at the top of the vertical air chambers, begin to inflate first, causing the chamber to further extend vertically. This feature creates a cradle around the occupant and prevents shifting once the vertical air chambers of the occupant's left side begin to inflate. Next, vertical air chambers on the occupant's left side continue to inflate causing the occupant's body to turn to the right, and, by release of pressure to an area external to the mattress, vertical air chambers on the occupant's immediate right begin to slowly deflate, allowing the occupant to be gently and gradually turned to a right-side position. As this sequence progresses, the control center will monitor the angle turned using contact sensors located on the top of the mattress. The vertical air chambers that are farthest from occupant's right side remain inflated so as to provide a barrier between occupant and the bed rails or to otherwise prevent occupant from moving beyond the mattress structure.

Now referring to FIG. 8A, a view in elevation showing the relative inflation of vertical air chambers in the upper pressurized compartment of the mattress structure when initiating a right turn rotation is depicted. FIG. 8B shows display window 755 on the control display 748 corresponding to the position of 8A. The display on the control center shows at all times, on various screens, the actions and positions of not only the vertical air chambers, but also a status each component of the mattress structure and its operation status. As each vertical air chamber reaches its required pressure (and height) in accordance with the programming, its corresponding bi-directional electronic air transfer valve will close, stopping the transfer of air to that chamber and allowing that chamber to maintain a constant pressure. When all the designated vertical air chambers reach their required or desired pressure point for the sequenced position—and provided that the occupant is actually in the position as programmed—the air compressor pump will not transfer additional air and the mattress structure will stabilize in that position at a constant pressure until it is time to transition to the next sequence.

At the beginning of the transition period for the next sequence, each bi-directional electronic air exchange valve will either slowly allow more air to enter its corresponding vertical air chamber or slowly release air from its vertical air chamber into the middle pressurized compartment until required pressure is reached in all vertical air chambers for the next position in the programmed sequence, as detected by the microelectronic remote sensors and directed by the electronic control center. The pressure in the middle-pressurized compartment will remain constant, even though during program intervals air will be exchanged through the bi-directional air pressure valves in the lower pressurized compartment. During any transition period where the net transfer of air is excessive than what the middle-pressurized compartment of the mattress structure requires, the excess air will be released through the release valve in the middle-pressurized compartment. Should there be a net deficiency of air during the transition period, the electric control center will cause the electric air compressor pump to supply additional air, which will filtrate to the component of the mattress structure in deficit as detected by the electronic control center.

The air exchange-stabilize processes will continue until all programmed sequences have been completed, which could include any combinations of the following features: degrees of turning, head or foot raising or lowering, massage, zero gravity, or any of the additional features noted below. Thus, the electronic control center directs the various electronic components of the mattress structure through all the programmed functions; and when complete, the mattress structure returns to the ‘baseline’ position for that occupant until further programmed.

Because of the design of the mattress structure and of the ability to program the movement of the vertical air chambers, the invention can provide those confined to or merely relaxing in bed with automatic adjustment benefits. In further embodiments, the frame of mattress structure—as done with conventional beds—is designed to assist with elevating and lowering the head and feet of the occupant, which dispenses with the need to adjust the hospital bed to extreme angles. In the event the hospital bed is adjusted to extreme angles, the lower pressurized compartment can be programmed to automatically compensate for the angles, allowing the airflow through the middle-pressurized compartment to remain possible.

In addition to the primary function of the mattress structure, the transition periods and rest periods are programmable. Transition periods may be programmed to be lengthened and the rests to be shortened in order to allow transitioning throughout a sequence or between sequences of a function to be continuous and virtually undetected by the most sensitive occupant. This function mimics a sense of being cradled and rocked. For quicker transition periods, the mattress structure may be programmed to provide appropriate periods of rest, where the vertical air chambers will maintain at a constant pressure in the programmed sequence for a set duration without changing, which allows the occupant to adjust from the quicker repositioning.

For the zero gravity operation or floating experiences, e.g., just after the mattress structure enters or returns to the baseline rest position for that occupant and while the occupant is on the mattress structure, the upper pressurized compartment consisting of the vertical air chambers may be pressurized slightly to provide a slight elevation off the mattress structure and to correspond to the occupant's weight distribution across the vertical air chambers, providing a zero gravity experience or the ‘equilibrium’ position while lying on the mattress structure. For the water motion or wave experience, while in a ‘equilibrium’ position, the pressure in the slightly pressurized vertical air chambers then can be randomly increased or decreased in small degrees, which will provide the sensation of floating. The electronic control center continually monitors the air pressure in all pressurized compartments to continue to give the occupant a zero gravity or floating experience, even when the occupant shifts or moves. Periods of equilibrium or floating may also be programmed within any programming sequence or function.

For operation for low pressure functions, the mattress structure may be programmed using the electronic control center to perform a preset or a specific pattern of raising and lowering the pressure within the vertical air chambers against the occupant's body or a certain part of an occupant's body. The random changing (or lowering) in pressure against the occupant's body prevent pressure sores and infections therefrom, and their resulting ailments. Because the low-pressure patterns may be controlled, they may be enabled to respond to music or to any other pattern while operating within a programmed sequence.

In embodiments, the air compressor pump is constructed as a permanent feature within the mattress structure; provided as a separate, but detachable item; or obtained by the user from of list of compatible models. Further, it is recognized that there are several types of pumps that may be used to service the invention, e.g., pneumatic, electric, hydraulic and pumps that that use fuels. Additionally, in alternative embodiments the medium pumped into the mattress components is air, other fluids may be used including water or oils. The air compressor pump may be constructed within the lower pressurized compartment of the mattress structure, anchored on the bed, or placed on the floor near the bed. Once activated, the electric air compressor pump provides the necessary air to inflate the mattress structure to the initial baseline rest position, as well as all other functions programmed by the user.

In operation, the electronic control center thereby instructs the electric air compressor pump to force air through the lower pressurized compartment, forces air into the open bi-directional air transfer valves, until the electronic control center detects that the proper pressure and position of the occupant has been reached, as read from the sensors; then the air forced by the electric air compressor pump stops. The mattress structure stabilizes. Next the weight of the occupant assists with the release of air from some of the vertical air chambers. The natural properties of the materials used in manufacturing the vertical air chambers further assist with the vertical air chambers returning to their natural size.

As described herein, it is apparent that the inflation sequence can be changed to achieve different objectives For example, once the mattress structure is in the full turn position and at rest, the vertical air chambers that are directly under the occupant's bed sores or injured areas can be programmed to decrease in pressure, allowing the occupant to stay in the programmed position but allowing minimal contact between the occupant and the mattress structure, thereby minimizing contact with certain pressure points.

Additional features that can be incorporated into the mattress include: (1) temperature, BP, and HR readings through the microelectronic remote sensors; (2) a Massage function—the low pressure function programmed as the increase and decrease of pressure in the vertical air chambers, resulting in a systematic wave across the width of the mattress structure or massages can be programmed as spikes in pressure within certain vertical air chambers located under the occupant; (3) a vibration function, and (4) an ocean (wave) function, which is similar to the massage function except that waves can be generated from various sections of the mattress structure resulting in a floating-rocking motion on water. Some features, particularly the last three, may be programmed to respond to and synchronize with music, beats or sound. Additionally, extra softness or firmness may be adjusted by the external mattress cover; and a cooling or heat mechanism may also be incorporated.

The suggested embodiments, operation and materials used are exemplary and are not to be restrictive of the invention. Finally, the accompanying drawings are attached hereto and incorporated herein and constitute a part of this application; and combined with the description, serve to describe and explain the principles of the invention in general terms and are not intended to be restrictive of the invention.

Contemplated further alternative embodiments include providing a series of air connection devices running along the top horizontal surface of the middle-pressurized compartment which may be bi-directional electronic air exchange valves, which are themselves are located inside each vertical air chambers of the upper pressurized compartment. In this embodiment, the equilibrium position of the mattress structure would be the zero-gravity position, as opposed to the base rest position in the preferred embodiment. The zero-gravity position is where the occupant should be placed on or removed from the mattress structure, or allowed to access the mattress or get off the mattress structure.

In a further alternative configuration, the bi-directional electronic air exchange valves are constructed to provide air with force, to propel or to jet air; much like the jets in hot tubs. Constructed in this manner, these valves would cause air exchange by force in and out of the middle-pressurized compartment and the vertical air chambers. This type of valve would provide for better massage feature. Yet another alternative embodiment, it may be adequate to only have one microelectronic remote sensor in each vertical air chamber, performing all monitoring tasks. As discussed above an alternative embodiment, each vertical air chamber may have two electronic air exchange valves—one allowing air to flow in the vertical air chambers and the other to flow out of the vertical air chambers.

Alternatively, the right and left sides of the upper pressurized compartments may be separate chambers divided down the center of the mattress structure from head to foot. Each side would operate independently of the other. The vertical air chambers in this embodiment would be better providing turns, particularly for those heavier occupants, but may be limited in providing other features.

In a different embodiment, the vertical air chambers in the upper pressurized compartment may be constructed of a harder gradually segmented material. These gradually segmented chambers will be airtight or enclosed in a lighter weight airtight material. As air enters each vertical air chamber and the pressure increases, the vertical air chamber telescopes out causing the chamber to expand in length (height) and the corresponding portion of the mattress structure will rise. Once the air pressure decreases, the telescoping effect will reverse. Because of the harder material, the vertical air chambers would not require a connection to the upper surface of the upper pressurized compartment.

Yet in another alternative embodiment, the middle-pressurized compartment's primary function may be to maintain sufficient pressure to allow inter-tubal networking and airducts to transfer air from the lower pressurized compartment to the upper pressurized compartment. The pressure of air in the tube associated with any vertical air chamber never changes, it is just funneled back and forth from the lower and upper pressurized compartments, with air being supplied by the electric air compressor pump. The detail of the tubal network depends on the types of feature(s) provided by the mattress structure. Additionally, the depth of the middle-pressurized compartment must accommodate the tubal networking.

Additionally, the lower pressurized compartment may be designed to assist with the handling of occupants with extreme weights. For example, the lower pressurized compartment may assist with turning the occupant by deflating or releasing air, allowing the gravity of the occupant t's weight to aide in making the turn resulting in a higher degree of rotation than capable by the vertical air chambers of the upper pressurized compartment working alone.

The advantage of the invention are apparent and include, (1) deep vein thrombosis mitigation (2) bed sore mitigation, (3) the promotion of healing and rehabilitation, (4) increase blood flow, (5) promotion of calmness to assist with some cognitive ailments, (6) provision of a level of pressure point relief, (7) pain relief, (8) provides physical comfort, (9) can assist with natural spine alignment, (10) can assist with low and gradual motion transfer and (1) promotion of emotional comfort and stability.

All descriptions, embodiments, illustrations, depictions, drawings, features, and features made herein are for the purpose of revealing the invention and are not intended to limit the scope and application of the invention which is defined in the appended claims.

I claim:

1. An inflatable mattress comprising:
a first section comprising an airtight chamber having a floor, a sidewall and a top layer, and a pressure valve, and said first section adapted to be filled with compressed air from an air pump through said valve, and
a second section comprised of a plurality of collapsible airtight chambers displaced across the first section and defining a top surface, wherein a least one chamber comprises a passage between said chamber and said first section and each said passage further comprises valves to control the flow of air in and out of said chamber, wherein a vertical dimension of said chamber changes in relation to the air pressure therein.

2. The mattress recited in claim 1 wherein said chambers are cylindrical and further comprise an air pressure release value and said air pressure release valve releases air external to said mattress.

3. The mattress recited of claim 1 further comprising comprises a central controller, a display, an air pump, and an input device, wherein and said pressure values are controlled by said controller and regulate the flow of air to and from said cylinder chambers, said display provides information relating to the inflation status of the mattress, and said air pump is controlled by said central controller to activate in response to instructions from said controller.

4. The mattress recited in claim 3 further comprising a memory, wherein said memory is adapted to be configured to store predetermined instructions that are executed by said controller to cause said chambers to inflate and deflate according to said instructions.

5. The mattress recited in claim 1, further comprising a third section wherein said third section supports said first section.

6. The mattress recited in claim 1 wherein said valve between said chambers and said first section is a bi-directional electronically controlled valve.

7. The mattress recited in claim 1 wherein central controller is programed to execute predetermined fill sequences of the chambers that make up said top section.

8. The mattress recited in claim 1 further comprising a plurality of pressure sensors for on the top surface at a plurality of locations across the top surface.

9. The mattress recited in claim 8 wherein said pressure sensors transmit signals to said controller and, in response to said transmitted signals, said controller sends output signals to said air pump and said pressure control values in response to said signals.

10. The mattress recited in claim 1 further comprising biometric sensors for the detection of biometric data and said sensors transmit said date to said central controller.

11. The mattress recited in claim 10 wherein said biometric sensors transmit signals to said controller and, in response to said transmitted biometric signals, said controller sends output signals to said air pump and said control values according to predetermined instructions.

12. An adjustable air mattress comprising a plurality of airtight elastic cylindrical chambers displaced across a support member, and each said cylinder further comprises at least one inlet passage to receive air and an exit vent value for the release of air pressure, and an air supply system, wherein said air supply system comprises an air pump, air hoses connecting said air pump to said cylindrical chambers and a plurality of electronically controlled valves associated with said hoses and controlled by a central controller.

13. The adjustable air mattress recited in claim 12 wherein said exit values are opened in response to a predetermined internal pressure threshold.

14. The adjustable air mattress of claim 12, wherein a valve is in communication with a supply hose that feeds a plurality of airtight cylindrical chambers and said plurality of chambers define a zone, and said controller control the inflation and deflation of said plurality of said chambers in said zone.

15. The adjustable air mattress of claim 14 comprise a plurality of zones and wherein said zones comprise linear arrangements of chambers that extended along the length of said mattress.

16. The adjustable mattress of claim 15 wherein said zones comprise a plurality of cylindrical chambers that are displaced laterally across the width of said matters.

17. The adjustable air mattress of claim 15 wherein said airtight cylindrical chambers comprise tubular elements, wherein said tubular elements expand longitudinally in response to an increase in air pressure in said chamber.

18. An adjustable air mattress of claim 17 wherein said tubular elements comprise an accordion arrangement that allows for the vertical displacement of said chamber in response to increased air pressure and restricts radial expansion.

19. The air mattress recited in claim 16 further comprising a central controller which adjusts the air flow to and from said chambers in said zones response to instructions and provides output to a display, and said display is adapted to display user input data and data from sensors associated with said mattress.

20. The air mattress recited in claim 12 further comprising a plurality of pressure sensors and said sensor detect the pressure applied to the top of said mattress and said pressure sensors are in communication with said controller and said controller is configured to adjustment said air pressure in said mattress in response to signals from said pressure sensors.

21. The air mattress recited in claim 12 further comprising biometric sensors and said biometric sensors are in communication with said controller and said controller is configured to adjustment said air pressure in said mattress in response to signals from said biometric sensors.
